(19) United States
(12) Patent Application Publication
(10) Pub. No.: US 2014/0194847 A1
(43) Pub. Date: Jul. 10, 2014

(54) TAMPON WITH NONPERMEABLE CAP

(71) Applicant: Lauren M. Adkins, Naples, FL (US)

(72) Inventor: Lauren M. Adkins, Naples, FL (US)

(21) Appl. No.: 14/154,879

(22) Filed: Jan. 14, 2014

(51) Int. Cl. A61F 13/20; A61F 13/34

(52) U.S. Cl. CPC A61F 13/208; A61F 13/34
USPC 604/385.17

(57) ABSTRACT

A tampon (1) having a nonpermeable cap (5) is placed over a distal end (4) of the tampon. The nonpermeable cap has a tubular wall (6) that surrounds the distal end of the tampon and rests inside the vagina (15). The tubular wall is pushed outward against the walls (16) of the vagina as the tampon expands with the absorption of fluid. The nonpermeable cap prevents liquids from escaping from the vagina or outside fluids being absorbed by the distal end of the tampon and into the vagina.

### FIELD OF THE INVENTION

This invention relates to tampons, and more particularly, to a tampon having a nonpermeable cap to prevent leaks.

### BACKGROUND OF THE INVENTION

A tampon is a mass of absorbent material used by women to insert into the vagina to to absorb the flow of menstrual fluid. Tampons are made of soft cotton pressed together to form a cylinder-like shape, so that they can be easily inserted into the opening of the vagina to absorb the menstrual flow before it has a chance to leave the body.

A problem with conventional tampons is that fluids can leak from the tampon onto undergarments and clothing. This can occur when the tampon becomes oversaturated and menstrual fluid either leaks from the end of the tampon and/or from a string attached to the end of the tampon. Another problem with conventional tampons is the absorption of fluids from outside the body, such as a pool or bath water. The absorption of outside fluids can be uncomfortable, cause the tampon to fail and leak and/or be unhygienic by introducing outside bacteria into the vagina.

Therefore, a need exists for a tampon having a nonpermeable cap that prevents leakage of menstrual fluid and prevents the absorption of outside fluids.

The relevant prior art includes the following references:

| Patent No. | Inventor | Issue/Publication Date |
|---|---|---|
| (U.S. Patent References) | | |
| 3,058,469 | Crockford | Oct. 16, 1962 |
| 3,499,448 | Jones | Mar. 10, 1970 |
| 6,177,608 | Weinstrauch | Jan. 23, 2001 |
| 6,939,333 | Franklin, Jr. | Sept. 06, 2005 |
| 2009/0281514 | Glaug et al. | Nov. 12, 2009 |
| (Foreign Patent References) | | |
| AU31177/71 | Glassman | Jan. 18, 1973 |

### SUMMARY OF THE INVENTION

The primary object of the present invention is to provide a tampon having a nonpermeable cap that prevents leakage of menstrual fluid and prevents the absorption of outside fluids.

The present invention fulfills the above and other objects by providing a tampon having a proximal end that is inserted into the vagina and rests near the cervix. A distal end of the tampon rests near the opening of the vagina. A nonpermeable cap is secured over the distal end of the tampon. The nonpermeable cap has a tubular wall that surrounds the distal end of the tampon and rests inside the vagina. The tubular wall is pushed outward against the walls of the vagina as the absorbent material of the tampon expands with the absorption of fluid. The nonpermeable cap prevents liquids from escaping from or being absorbed by the distal end of the tampon. A string, knob or nipple extends from the base of the nonpermeable cap to assist a female in removing the tampon from the vagina. The string, knob or nipple preferably has no contact with the absorbent material of the tampon, thereby preventing any fluid from seeping through the string, knob or nipple either from or to the absorbent material of the tampon.

The above and other objects, features and advantages of the present invention should become even more readily apparent to those skilled in the art upon a reading of the following detailed description in conjunction with the drawings wherein there is shown and described illustrative embodiments of the invention.

### BRIEF DESCRIPTION OF THE DRAWINGS

In the following detailed description, reference will be made to the attached drawings in which:

FIG. 1 is a side view of a tampon of the present invention;

FIG. 2 is a cross-sectional view along line 2-2 of FIG. 1 of a tampon of the present invention;

FIG. 3 is a side view of a tampon of the present invention having a nipple located on the non-permeable cap;

FIG. 4 is a side plan view of a tampon of the present invention located in an applicator; and

FIG. 5 is a view of a tampon of the present invention inserted into a vagina and in use.

### DESCRIPTION OF THE PREFERRED EMBODIMENTS

For purposes of describing the preferred embodiment, the terminology used in reference to the numbered components in the drawings is as follows:
1. tampon, generally
2. main body
3. proximal end of the main body
4. distal end of the main body
5. nonpermeable cap
6. tubular wall of nonpermeable cap
7. end wall of nonpermeable cap
8. upper perimeter edge of tubular wall
9. removal aid
10. extension
11. nipple
12. applicator
13. outer tube
14. inner tube
15. vagina
16. vaginal wall

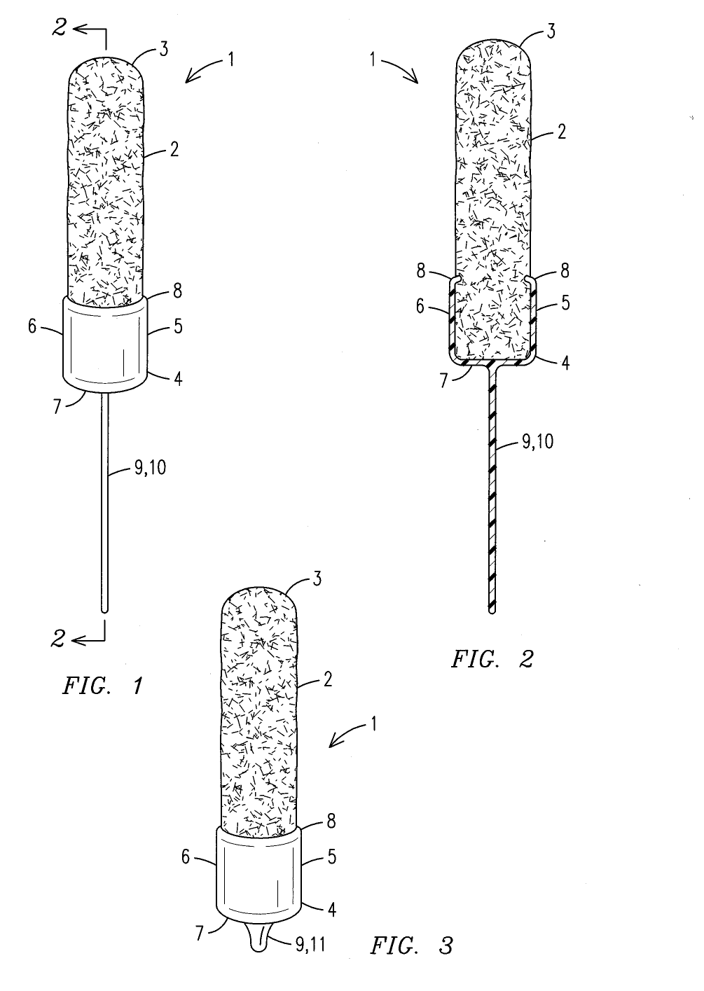

With reference to FIG. 1 and FIG. 2, a side view of a tampon 1 of the present invention and a cross-sectional view along lines 2-2 of FIG. 1 of a tampon 1 of the present invention, respectively, are illustrated. The tampon 1 comprises a substantially cylindrically-shaped main body 2 constructed from an absorbent material and having a proximal end 3 and a distal end 4. A nonpermeable cap 5 is placed over the distal end 4 of the tampon 1. The nonpermeable cap 5 has a tubular wall 6 and an end wall 7 that surrounds the distal end 4 of the tampon 1. The nonpermeable cap 5 is preferably constructed from a nonpermeable or fluid resistant material that is also stretchable and/or has elastomeric qualities, such as rubber, latex, vinyl and so forth. The nonpermeable cap 5 may be attached to the main body 2 of the tampon 1 by an attachment means, such as adhesive, to secure the nonpermeable cap 5 to the absorbent material of the main body 2.

In addition, an upper perimeter edge 8 of the tubular wall 6 of the nonpermeable cap 5 may be angled inward against the main body 2 of the tampon 1 to further prevent any separation of the nonpermeable cap 5 from the main body 2 of the tampon 1. The angled upper perimeter edge 8 of the main body 2 of the tampon 1 also provides additional comfort when inserting and wearing the tampon 1 by preventing the upper perimeter edge 8 from rubbing against and irritating the walls of the vagina.

A removal aid 9, such as an extension 10 of the nonpermeable cap or string, is located on the nonpermeable cap 5 to assist a female in removing the tampon 1 from the vagina. As illustrated here, the removal aid 9 is an extension 10 constructed from a nonpermeable material extending from the end wall 7 of the nonpermeable cap 5. The removal aid 9 preferably has no contact with the absorbent material of the main body 2 of the tampon 1, thereby preventing and fluid from seeping through the removal aid 9 either from or to the absorbent material of the tampon 1.

With reference to FIG. 3, a side view of a tampon 1 of the present invention having a nipple 11 for use as a removal aid 9 located on the non-permeable cap 5 is illustrated. The nipple 11 is located on the nonpermeable cap to assist a female in removing the tampon 1 from the vagina. The removal aid 9 preferably has no contact with the absorbent material of the main body 2 of the tampon 1, thereby preventing and fluid from seeping through the removal aid 9 either from or to the absorbent material of the tampon 1.

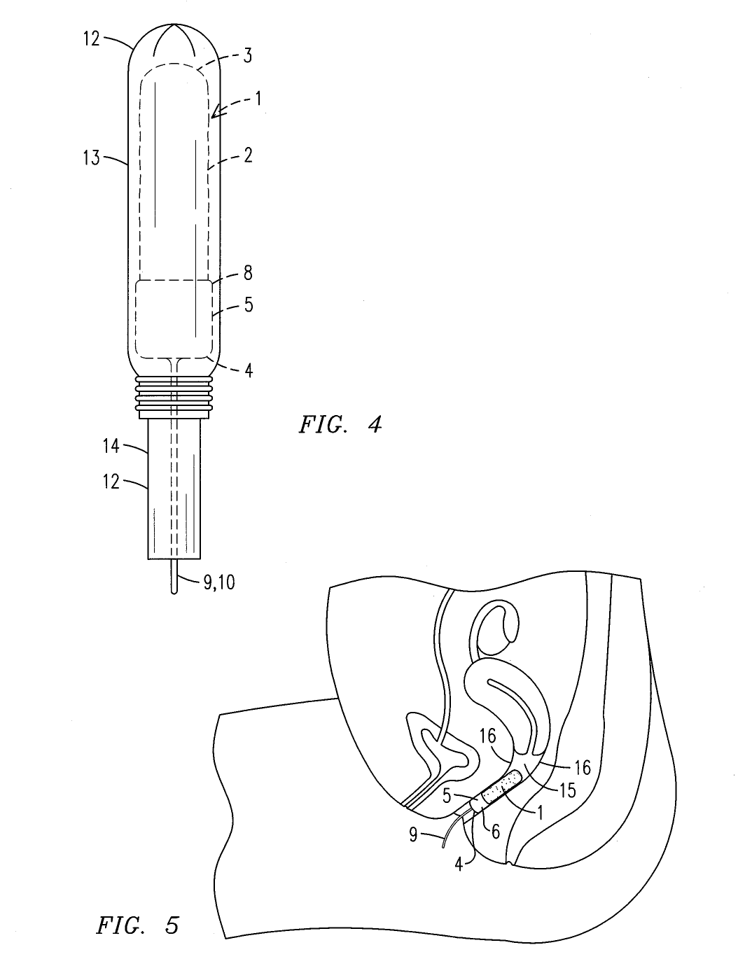

With reference to FIG. 4, a side plan view of a tampon 1 of the present invention in an applicator 12 is illustrated. The tampon 1 is located within an example of a conventional applicator 12 used for inserting the tampon 1 into a vagina. The applicator 12 comprises an outer tube 13 and an inner tube 14. The tampon 1 is located within the outer tube 13 which is placed within the vagina. The inner tube 14 is then used to push the tampon 1 upward and out of the outer tube 13 into the vagina as the outer tube 13 is simultaneously removed from the vagina.

With reference to FIG. 5, a view of a tampon 1 of the present invention inserted into a vagina 15 and in use. The nonpermeable cap 5 has a tubular wall 6 that surrounds the distal end 4 of the tampon 1 and rests inside the vagina 15. The tubular wall 6 is pushed outward against the walls 16 of the vagina 15 as the absorbent material of the main body 2 of the tampon 1 expands with the absorption of fluid. The nonpermeable cap 5 prevents liquids from escaping from or being absorbed by the distal end 4 of the tampon 1.

It is to be understood that while a preferred embodiment of the invention is illustrated, it is not to be limited to the specific form or arrangement of parts herein described and shown. It will be apparent to those skilled in the art that various changes may be made without departing from the scope of the invention and the invention is not to be considered limited to what is shown and described in the specification and drawings.

Having thus described my invention, I claim:

1. A tampon comprising:
a substantially cylindrically-shaped main body constructed from an absorbent material;
said main body having a proximal end and a distal end;
a nonpermeable cap having a tubular wall and an end wall; and
said nonpermeable cap being placed over the distal end of the main body so the tubular wall of the nonpermeable cap surrounds the distal end of the main body to prevent the leakage of fluids through the distal end of the main body.

2. The tampon of claim 1 further comprising:
an attachment means to secure the nonpermeable cap to the absorbent material of the main body to prevent any separation of the nonpermeable cap from the main body of the tampon.

3. The tampon of claim 1 wherein:
an upper perimeter edge of the tubular wall of the nonpermeable cap is angled inward against the main body of the tampon to prevent any separation of the nonpermeable cap from the main body of the tampon.

4. The tampon of claim 1 further comprising:
a removal aid located on the nonpermeable cap to assist a female in removing the tampon from the vagina.

5. The tampon of claim 4 wherein:
the removal aid is separated from the main body of the tampon by the nonpermeable cap.

6. The tampon of claim 4 wherein:
the removal aid is an extension.

7. The tampon of claim 4 wherein:
the removal aid is a nipple.

8. A tampon comprising:
a substantially cylindrically-shaped main body constructed from an absorbent material;
said main body having a proximal end and a distal end;
a nonpermeable cap having a tubular wall and an end wall;
said nonpermeable cap being placed over the distal end of the main body so the tubular wall of the nonpermeable cap surrounds the distal end of the main body to prevent the leakage of fluids through the distal end of the main body; and
an upper perimeter edge of the tubular wall of the nonpermeable cap is angled inward against the main body of the tampon to prevent any separation of the nonpermeable cap from the main body of the tampon.

9. The tampon of claim 8 further comprising:
an attachment means to secure the nonpermeable cap to the absorbent material of the main body to prevent any separation of the nonpermeable cap from the main body of the tampon.

10. The tampon of claim 8 further comprising:
a removal aid located on the nonpermeable cap to assist a female in removing the tampon from the vagina.

11. The tampon of claim 10 wherein:
the removal aid is separated from the main body of the tampon by the nonpermeable cap.

12. The tampon of claim 10 wherein:
the removal aid is an extension.

13. The tampon of claim 10 wherein:
the removal aid is a nipple.

14. A tampon comprising:
a substantially cylindrically-shaped main body constructed from an absorbent material;
said main body having a proximal end and a distal end;
a nonpermeable cap having a tubular wall and an end wall;
said nonpermeable cap being placed over the distal end of the main body so the tubular wall of the nonpermeable cap surrounds the distal end of the main body to prevent the leakage of fluids through the distal end of the main body; and
a removal aid located on the nonpermeable cap to assist a female in removing the tampon from the vagina.

15. The tampon of claim 14 further comprising:
an attachment means to secure the nonpermeable cap to the absorbent material of the main body to prevent any separation of the nonpermeable cap from the main body of the tampon.

16. The tampon of claim 14 wherein:
an upper perimeter edge of the tubular wall of the nonpermeable cap is angled inward against the main body of the tampon to prevent any separation of the nonpermeable cap from the main body of the tampon.

17. The tampon of claim 14 wherein:
the removal aid is separated from the main body of the tampon by the nonpermeable cap.

18. The tampon of claim 14 wherein:
the removal aid is an extension.

19. The tampon of claim 14 wherein:
the removal aid is a nipple.
